(19) United States
(12) Patent Application Publication
(10) Pub. No.: US 2012/0330349 A1
(43) Pub. Date: Dec. 27, 2012

(54) IMPLANT DELIVERY AND ACTIVE RELEASE SYSTEM

(76) Inventors: Donald K. Jones, Dripping Springs, TX (US); Vladimir Mitelberg, Austin, TX (US)

(21) Appl. No.: 13/533,592

(22) Filed: Jun. 26, 2012

Related U.S. Application Data
(60) Provisional application No. 61/501,667, filed on Jun. 27, 2011.
(60) Provisional application No. 61/501,682, filed on Jun. 27, 2011.

(51) Int. Cl. A61M 29/00

(52) U.S. Cl. 606/200

(57) ABSTRACT

A medical implant deployment system for placing an implant at a preselected site within a vessel, duct or body lumen of a mammal. The deployment system includes a heating element at the distal end of a positioning member and a thermal responsive coupling including a thermal responsive element connected to the implant by a frangible member. After positioning the implant, the heating element is activated which causes the thermal responsive element to apply mechanical force sufficient to break the frangible member using traction, thereby releasing the implant at a desired position within the body.

### CROSS-REFERENCE TO RELATED APPLICATIONS

This application claims the benefit of U.S. Prov. Ser. 61/501,667 filed Jun. 27, 2011 and U.S. Prov. Ser. 61/501,682 filed Jun. 27, 2011, all of which are hereby incorporated by reference herein in their entireties.

### BACKGROUND OF THE INVENTION

For many years flexible catheters have been used to place various devices within the vessels of the human body. Such devices include dilatation balloons, radio-opaque fluids, liquid medications and various types of occlusion devices such as balloons and embolic coils. Examples of such catheter devices are disclosed in U.S. Pat. No. 5,108, 407, entitled “Method And Apparatus For Placement Of An Embolic Coil”; U.S. Pat. No. 5,122,136, entitled, “Endovascular Electrolytically Detachable Guidewire Tip For The Electroformation Of Thrombus In Arteries, Veins, Aneurysms, Vascular Malformations And Arteriovenous Fistulas.” These patents disclose devices for delivering embolic coils to preselected positions within vessel of the human body in order to treat aneurysms, or alternatively, to occlude the blood vessel at the particular location.

Coils which are placed in vessels may take the form of helically wound coils, or alternatively, may be random wound coils, coils wound within other coils or many other such configurations. Examples of various coil configurations are disclosed in U.S. Pat. No. 5,334,210, entitled, “Vascular Occlusion Assembly; U.S. Pat. No. 5,382,259, entitled, “Vasoocclusion Coil With Attached Tubular Woven Or Braided Fibrous Coverings.” Embolic coils are generally formed of radiopaque metallic materials, such as platinum, gold, tungsten, or alloys of these metals. Often times, several coils are placed at a given location in order to occlude the flow of blood through the vessel by promoting thrombus formation at the particular location.

In the past, embolic coils have been placed within the distal end of the catheter. When the distal end of the catheter is properly positioned the coil may then be pushed out of the end of the catheter with, for example, a guidewire to release the coil at the desired location. This procedure of placement of the embolic coil is conducted under fluoroscopic visualization such that the movement of the coil through the vasculature of the body may be monitored and the coil may be placed at the desired location. With these placements systems there is very little control over the exact placement of the coil since the coil may be ejected to a position some distance beyond the end of the catheter.

Numerous procedures have been developed to enable more accurate positioning of coils within a vessel. Still another such procedure involves the use of a glue, or solder, for attaching the embolic coil to a guidewire which, is in turn, placed within a flexible catheter for positioning the coil within the vessel at a preselected position. Once the coil is at the desired position, the coil is restrained by the catheter and the guidewire is pulled from the proximal end of the catheter to thereby cause the coil to become detached from the guidewire and released from the catheter system. Such a coil positioning system is disclosed in U.S. Pat. No. 5,263,964, entitled, “Coaxial Traction Detachment Apparatus And Method.”

Another coil positioning system utilizes a catheter having a socket at the distal end of the catheter for retaining a ball which is bonded to the proximal end of the coil. The ball, which is larger in diameter than the outside diameter of the coil, is placed in a socket within the lumen at the distal end of the catheter and the catheter is then moved into a vessel in order to place the coil at a desired position. Once the position is reached, a pusher wire with a piston at the end thereof is pushed distally from the proximal end of the catheter to thereby push the ball out of the socket in order to release the coil at the desired position. Such a system is disclosed in U.S. Pat. No. 5,350, 397, entitled, “Axially Detachable Embolic Coil Assembly.” One problem with this type of coil placement system which utilizes a pusher wire which extends through the entire length of the catheter and which is sufficiently stiff to push an attachment ball out of engagement with the socket at the distal end of the catheter is that the pusher wire inherently causes the catheter to be very stiff with the result that it is very difficult to guide the catheter through the vasculature of the body.

Another method for placing an embolic coil is that of utilizing a heat releasable adhesive bond for retaining the coil at the distal end of the catheter. One such system uses laser energy which is transmitted through a fiber optic cable in order to apply heat to the adhesive bond in order to release the coil from the end of the catheter. Such a method is disclosed in U.S. Pat. No. 5,108,407, entitled, “Method And Apparatus For Placement Of An Embolic Coil.” Such a system also suffers from the problem of having a separate, relatively stiff element which extends throughout the length of the catheter with resulting stiffness of the catheter.

Another method for placing an embolic coil is that of utilizing a heat responsive coupling member which bonds the coil to the distal end of a delivery system. One such system uses electrical energy which is transmitted through electrical conductors to create heat which is applied to the coupling member to thereby soften and yield the coupling member in order to release the coil from the end of the delivery system. Such a method is disclosed in U.S. Pat. No. 7,179,276, entitled, “Heated Vascular Occlusion Coil Deployment System.” Such a system suffers from the problem of having to pull an engagement member once the coupling is softened in order to release the coil.

### SUMMARY OF THE INVENTION

The present invention is directed toward a medical implant deployment system for use in placing a medical implant at a preselected site within the body of a mammal which includes an elongated flexible positioning member having a lumen extending therethrough. A thermal responsive coupling member having a tubular housing, a heating element and a thermal responsive element positioned within the housing is fixedly located at the distal end of the positioning member. The thermal responsive element having two ends is fixedly bonded at one end to the housing and the other end coupled to the implant by a frangible member. The heating element is adapted to be coupled to a source of energy through an energy transmission conductor which extends from the proximal end of the positioning member to the distal end of the positioning member. The thermal responsive element, connecting the implant to the positioning member has a first axial length and exhibits the characteristic of contracting in axial length upon being heated. When energy is applied through the conductor to the heating element, the heating element causes the thermal responsive element to contract and apply a tensile load to the proximal end of the implant. Movement of the proximal end of the implant is restricted by the housing of the thermal responsive coupling thereby causing the tensile load generated by the contracting thermal responsive element to be applied to the frangible member bonded to the implant. When the tensile load applied to the frangible member exceeds the yield strength of the frangible member or the bond strength coupling the implant to the thermal responsive element, the frangible member breaks or breaks away from the implant thereby releasing implant under traction at a preselected site within the body of a mammal.

In accordance with another aspect of the present invention, the energy transmission conductor takes the form of two electrical conductors which extend from the proximal end of the positioning member and are connected to the heating element coil for applying electrical energy to the heating element to thereby cause the thermal responsive element to become heated.

In accordance with an aspect of the present invention the heating element takes the form of a resistive heating coil. At least a portion of the heating coil is secured to the housing to thereby apply heat energy to the thermal responsive element when the heating coil is supplied with electrical energy.

In accordance with another aspect of the present invention, the heating element is integrally formed with the thermal responsive element such that supplying electrical energy to the thermal responsive element causes the thermal responsive element to resistively heat thereby initiating contraction of the thermal responsive element.

In accordance with still another aspect of the present invention the thermal responsive element is formed of a material susceptible to substantial dimensional changes of between 2 to 10 percent with respect to preset temperatures. Suitable materials include some shape memory materials formed of polymers and metal alloys such as nitinol.

In accordance yet another aspect of the present invention, the frangible member takes the form of a material exhibiting the characteristic of having a low percent elongation under tensile loading prior to fracture. Suitable materials should have an elongation less than about 5% and preferably less than 2%. These materials include metal alloys such as solder and polymers such as cyanoacrylates, epoxies and UV adhesives.

In accordance with one aspect of the present invention, the medical implant takes the form of an embolic coil for selective placement within a vessel, aneurysm, duct or other body lumen.

In accordance with yet still another aspect of the present invention, the medical implant may take the form of a stent for selective placement within a vessel, duct or other body lumen.

These aspects of the invention and the advantages thereof will be more clearly understood from the following description and drawings of a preferred embodiment of the present invention:

### BRIEF DESCRIPTION OF THE DRAWINGS

FIG. 1 is an enlarged, partially sectioned view of the medical implant deployment system of the present invention;

FIG. 2 is an enlarged partially sectioned view showing the medical implant deployment system prior to placement within a catheter;

FIGS. 3 through 5 are enlarged partially sectioned views illustrating the sequential steps of positioning an embolic coil implant within a vessel and releasing the coil at a preselected site.

### DETAILED DESCRIPTION OF THE INVENTION

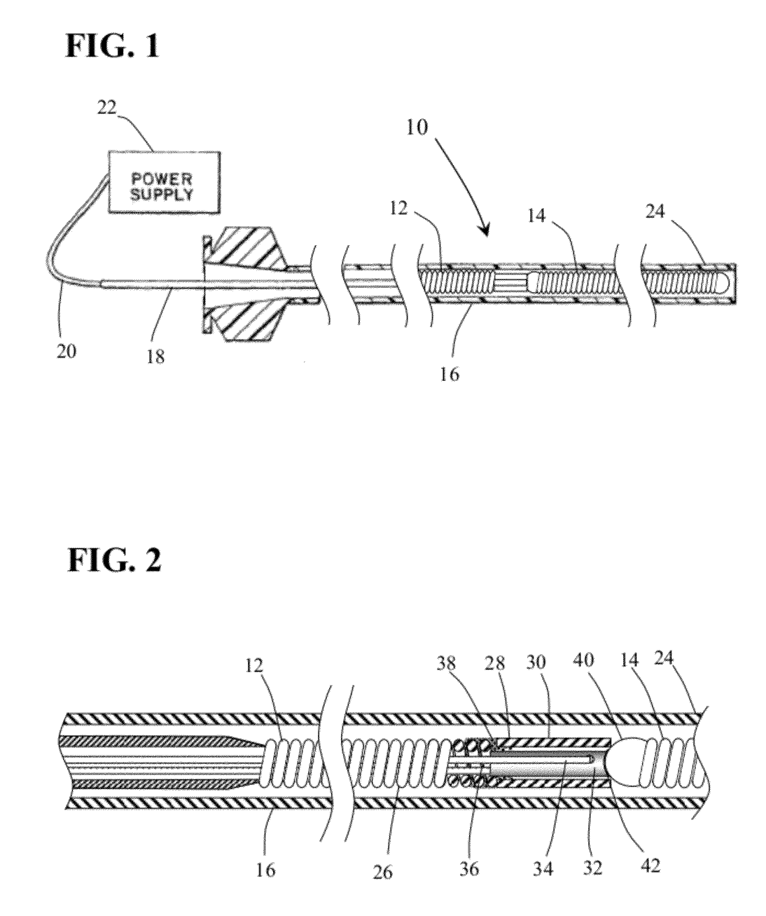

Generally a medical implant deployment system of the present invention may be used to position an implant at a preselected site within the body of a mammal. The medical implant deployment system may be used to place various implants such as stents, filters and embolization coils. FIG. 1 generally illustrates a medical implant deployment system 10 of the present invention which includes an elongate positioning member 12 coupled to an implant such as an embolization coil 14 for vascular occlusion. Embolization coil 14 is of a general type suitable for use in occluding a vessel, duct or aneurysm. The coil is generally formed of a helically wound wire material which is biocompatible and preferably radio-opaque. Typical metallic materials for the coil include platinum alloys, gold, tantalum and stainless steel. Biocompatible polymeric materials may also be suitable. The coil may include fibers or bioactive materials to improve the embolization process when implanted. The coils may also have secondary shapes which improve their suitability for various anatomical geometries. The positioning member 12 and coil 14 are slidably positioned within the lumen of flexible catheter 16. The proximal end 18 of positioning member 12 extends from the proximal end of catheter 16. An energy cable 20 extends from the proximal end of positioning member 12 to a power supply 22.

FIG. 2 illustrates in more detail the construction of the implant deployment system with the coil 14 being positioned within the lumen of the catheter distal end 24. More particularly, the implant deployment system includes an elongated positioning member 12 which generally has a length longer than catheter 16. Positioning member 12 has a lumen extending from its proximal end to its distal end 26. Located adjacent distal end 26 of the positioning member is a thermal responsive coupling 28. The thermal responsive coupling includes a tubular coupling housing 30, a thermal responsive element 32 and a pair of energy transmission conductors 34 and 36 affixed to thermal responsive element 32. While the thermal responsive coupling may include a separate heating element, preferably formed as a resistively heated coil, the present configuration of thermal responsive element 32 also serves as an integrally formed heating element. Thermal responsive element 32 having proximal and distal ends is positioned within tubular housing 30 where the proximal end is immovably secured to housing 30 using fixation member 38. The fixation member may take the form of any material or process suitable for fixedly securing the proximal end of the thermal responsive element relative to the proximal end of housing 30 such as solder, epoxy or welding. The thermal responsive coupling 28 is fixedly secured to the distal end of positioning member 12. The distal end thermal responsive element 32 is coupled to coil 14 at its proximal end 40 by frangible member 42. The proximal end 40 of coil 14 is a preferably formed as a rounded atraumatic bead of the coil using well known beading processes. Alternatively, the proximal end 40 may be an added structure which is secured to the coil. The proximal end 40 has a diameter which is larger than the lumen of the housing 30 thereby preventing coil 14 from becoming lodged within the housing. The thermal responsive element may take the form of any biocompatible shape memory material (metals or polymers) that may change its length upon exposure to heat. Preferably, the thermal responsive element 32 comprises a muscle wire such as, for example, a nickel titanium shape memory alloy manufactured by Dynalloy, Inc. sold under the trademarked name Flexinol.

Also, as illustrated in FIG. 2, the heating element and integrally formed thermal responsive element 32 is coupled to a pair of energy transmission conductors 34 and 36. Preferably the energy transmission conductors 34 and 36 are electrical conductors also coupled to power supply 22 via cable 20. Upon application of an electrical current to the pair of conductors 34 and 36, the thermal responsive element 32 begins to resistively heat to thereby cause the thermal responsive element 32 to actuate. As the thermal responsive element 32 heats it shortens in length thus bringing the proximal end 40 of coil 14 into contact with the distal end of housing 30. A tensile load is generated on frangible member 42 connecting thermal responsive element 32 to coil 14. Once the generated tensile force exceeds the yield strength of frangible member 42 or the bond strength to proximal end 40, the frangible member breaks from proximal end 40 thereby releasing coil 14.

The frangible member 42 may take the form of any biocompatible bonding agent which has the characteristic of having a low percent elongation when placed under tensile loading. Suitable materials include metals such as solder and polymer adhesives such as cyanoacrylates, UV adhesives and epoxies. Polymers and blends such as polystyrene and ABS (poly (acrylonitrile butadiene styrene)) may be configured to serve as a frangible member.

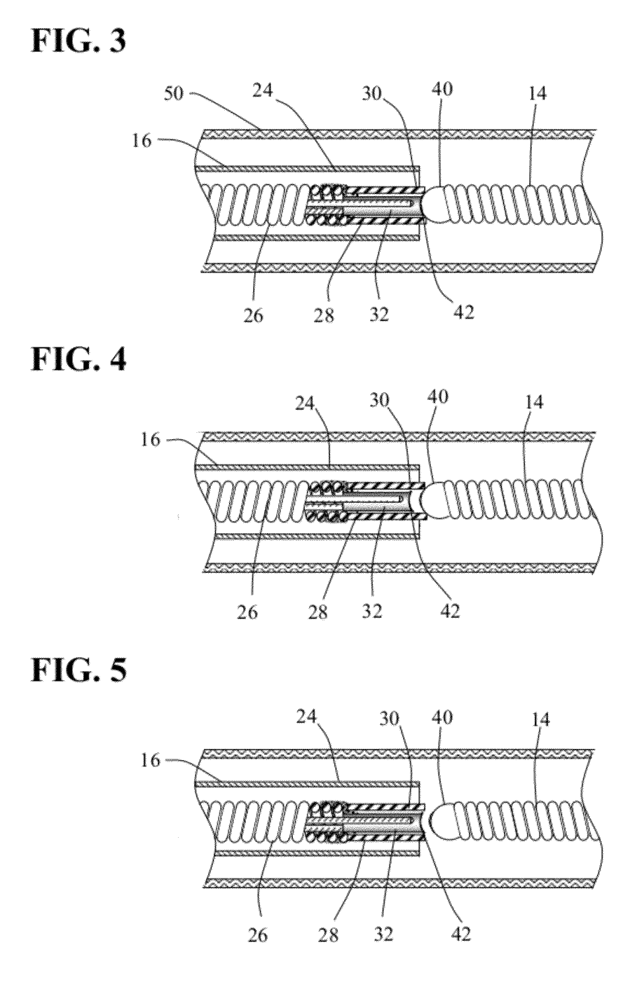

More particularly, and as illustrated in FIGS. 3 through 5, the implant deployment system 10 is inserted into a blood vessel 50 and is moved to a position within the blood vessel 50 to a position where it is desirable to place the embolic coil 14. When the catheter 16 has been positioned at a location slightly proximal of the preselected site for placement of the embolic coil (FIG. 3), the positioning member 12 is pushed out of the distal end of the catheter 16 and electrical energy is then applied to the thermal responsive element 32 to thereby cause the thermal responsive element 32 to contract. As the thermal responsive member 32 retracts relative to housing 30, movement of the proximal end 40 of embolic coil 14 is restricted by housing 30 thus causing the frangible member 42 to break thereby releasing the coil 14 (FIG. 4). As shown in FIG. 5, once the electrical energy is discontinued the thermal responsive element 32 may return to its normal length, however, as the positioning member 12 and catheter 16 are retracted there is no longer engagement between the thermal responsive coupling 28 and the embolic coil 14 and the coil remains deposited at the desired location.

With the implant deployment system of the present invention it is possible to place an embolic coil very precisely at a desired location within a vessel. Once the coil has been placed in a preselected location by catheter, the deployment system may be activated by applying energy to a thermal responsive coupling to thereby cause the coil to be released and deposited at a desired location.

As is apparent, there are numerous modifications of the preferred embodiment described above which will become readily apparent to one skilled in the art, such as many variations and modifications of the deployment system including many different variations of the heating element, many variations of energy sources for heating the adhesive such as optical, radiofrequency, and acoustical, many variations of energy transmission conductors such as optical fiber, and many variations of frangible members.

These modifications would be apparent to those having ordinary skill in the art to which this invention relates and are intended to be within the scope of the claims which follow.

That which is claimed is:

1. A vascular occlusion coil deployment system for use in placing a coil at a preselected site within a vessel comprising:
an elongate flexible positioning member having a lumen extending therethrough and having proximal and distal ends;
an embolic coil having proximal and distal ends;
a thermal responsive coupling member mounted on the distal end of said positioning member, said coupling member having a housing member, a heating element, a thermal responsive element having proximal and distal ends and a frangible member coupled to and positioned between said thermal responsive element distal end and said embolic coil,
said thermal responsive element having a first configuration coupled to said embolic coil and a second configuration separated from said embolic coil by traction wherein said thermal responsive element is operable between said first and second configurations upon the application of energy to said responsive element;
an energy transmission conductor extending through the lumen of the positioning member and extending from the proximal end to the distal end of the positioning member, said energy transmission conductor being coupled to said thermal responsive coupling member; and
a power supply adapted to provide energy to said energy transmission conductor such that upon the application of energy to said energy transmission conductor said heating element heats thereby causing said thermal responsive element to move from said first configuration to said second configuration and release said embolic coil.

2. A vascular occlusion coil deployment system as defined in claim 1, wherein said thermal responsive element comprises a shape memory material.

3. A vascular occlusion coil deployment system as defined in claim 2, wherein said shape memory material is nitinol.

4. A vascular occlusion coil deployment system as defined in claim 1, wherein said heating element and said thermal responsive element are integrally formed.

5. A vascular occlusion coil deployment system as defined in claim 4, wherein said frangible member comprises a polymer.

6. A vascular occlusion coil deployment system as defined in claim 4, wherein said frangible member comprises a solder.

7. A vascular occlusion coil deployment system for use in placing a coil at a preselected site within the body of a mammal comprising:
an elongated flexible positioning member having proximal and distal ends;
an embolic coil having proximal and distal ends;
a thermal responsive coupling member mounted on the distal end of said positioning member, said coupling member having a tubular housing member having proximal and distal ends and a thermal responsive element positioned within the lumen of said tubular housing member having proximal and distal ends wherein the distal end of said thermal responsive element is coupled to said embolic coil and said embolic coil is positioned distal to said thermal responsive element,
said thermal responsive element having a first axial length when said element distal end is coupled to said embolic coil and a second axial length different from said first length, said thermal responsive element is operable between said first and second axial lengths upon the application of energy to said thermal responsive element;
an elongate energy transmission conductor having proximal and distal ends and extends from the proximal end of the positioning member to the distal end of the positioning member, said energy transmission conductor distal end is coupled to said thermal responsive element; and
a power supply adapted to provide energy to said energy transmission conductor such that upon the application energy to said energy transmission conductor proximal end said thermal responsive element moves from said first axial length to said second axial length to thereby uncouple said thermal responsive element distal end from said embolic coil.

8. A vascular occlusion coil deployment system as defined in claim 7, wherein said energy transmission conductor comprises a pair of electrical energy conductors and said thermal responsive element is resistively heated.

9. A vascular occlusion coil deployment system as defined in claim 8, wherein said thermal responsive element second axial length is smaller than said first axial length.

10. A vascular occlusion coil deployment system as defined in claim 8, wherein said thermal responsive element comprises shape memory material.

11. A vascular occlusion coil deployment system as defined in claim 10, wherein said shape memory material is nitinol.

12. A medical implant deployment system for use in placing an implant at a preselected site within the body comprising:
an elongate flexible positioning member having proximal and distal ends;
an implant having proximal and distal ends;
a thermal responsive coupling member mounted on the distal end of said positioning member, said coupling member having a housing member, a heating element, a thermal responsive element having proximal and distal ends and a frangible member coupled to and positioned between said thermal responsive element distal end and said implant,
said thermal responsive element having a first configuration coupled to said implant and a second configuration uncoupled from said implant whereby uncoupling of said implant occurs by traction, said thermal responsive element being operable between said first and second configurations upon the application of energy to said thermal responsive element;
an energy transmission conductor extending from the proximal end of said positioning member to the distal end of said positioning member, said energy transmission conductor being coupled to said thermal responsive coupling member; and
a power supply adapted to provide energy to said energy transmission conductor such that upon the application of energy to said energy transmission conductor said heating element heats thereby causing said thermal responsive element to move from said first configuration to said second configuration and release said implant.

13. A medical implant deployment system as defined in claim 12, wherein said thermal responsive element comprises a shape memory material.

14. A medical implant deployment system as defined in claim 13, wherein said shape memory material is nitinol.

15. A medical implant deployment system as defined in claim 12, wherein said frangible member comprises a polymer.

16. A medical implant deployment system as defined in claim 12, wherein said frangible member comprises a solder.

17. A medical implant deployment system as defined in claim 12, wherein said heating element and said thermal responsive element are integrally formed.

18. A medical implant deployment system as defined in claim 17, wherein said thermal responsive element comprises a shape memory material

19. A medical implant deployment system as defined in claim 18, wherein said frangible member comprises a polymer.

20. A medical implant deployment system as defined in claim 18, wherein said frangible member comprises a solder.
